(19) United States
(12) Patent Application Publication
(10) Pub. No.: US 2006/0129082 A1
(43) Pub. Date: Jun. 15, 2006

(54) Selective plasma exchange therapy

(76) Inventor: Jacek Rozga, Beverly Hills, CA (US)

Correspondence Address: EDWARDS & ANGELL, LLP, P.O. BOX 55874, BOSTON, MA, 02205, US

(21) Appl. No.: 10/524,448

(22) PCT Filed: Aug. 11, 2003
(86) PCT No.: PCT/US03/25162

§ 371 Date: Sep. 29, 2005

Related U.S. Application Data
(60) Provisional application No. 60/403,099, filed on Aug. 13, 2002.

(51) Int. Cl. A61M 37/00

(52) U.S. Cl. 604/6.04

(57) ABSTRACT

Disclosed is a method for removing from a patient's blood a specific plasma fraction containing substances within a specific molecular weight range and a plasma purification apparatus by which the method can be accomplished.

### BACKGROUND OF INVENTION

1. Field of the Invention

The present invention relates to the medical arts, and in particular to blood purification therapy.

2. Discussion of the Related Art

In many diseases and pathological conditions such as liver failure, familial hypercholesterolemia, and sepsis there is an accumulation of specific substances in the circulating blood that cause harm and should be removed. There are a number of ways by which circulating blood has been purified of toxic substances, including: blood/plasma sorption therapy, cascade plasma filtration, and whole plasma exchange therapy.

Blood/plasma sorption therapy is performed either directly on whole blood or plasma, or coupled with hemodialysis/hemofiltration to treat either the dialysate or hemofiltrate. (Kiley J E, Welch H F, Pender J C. Removal of blood ammonia by hemodialysis. Proc Soc Exp Biol Med 1956; 91: 489-90; Shibusawa K, Tago J. Artificial kidney. Saishin-igaku 1956; 11: 298-310; Chang T M S. Hemoperfusion over microencapsulated adsorbent in a patient with hepatic coma. Lancet 1972; 2: 1371; Silk DBA, Trewby P N, Chase R A, et al. Treatment of fulminant hepatic failure by polyacrylonitrile-membrane haemodialysis. Lancet 1977; 2: 1-3; Denis J, Opolon P, Nusinovici V, et al. Treatment of encephalopathy during fulminant hepatic failure by haemodialysis with high permeability membrane. Gut 1978; 19: 787-93; Gimson A E S, Mellon P J, Braude S, et al. Earlier charcoal haemoperfusion in fulminant hepatic failure. Lancet 1982; 2: 681-83; Denis J, Opolon P, Delorme M-L. Long-term extra-corporeal assistance by continuous haemofiltration during fulminant hepatic failure. Gastroenterol Clin Biol 1979; 3: 337-48; Matsubara S, Okabe K, Ouchi K, et al. Continuous removal of middle molecules by hemofiltration in patients with acute liver failure. Crit Care Med 1990; 18: 1331-38).

None of the therapeutic modalities of blood/plasma sorption therapy used to date has achieved wide clinical use or ability to arrest or reverse liver failure and improve survival. Furthermore, the repertoire of putative toxins of hepatic coma is large and includes not only small substances such as ammonia, phenols, mercaptans, false neurotransmitters, aromatic amino acids, short-chain fatty acids, but also abnormal “middle” molecules (MW 5 kDa to 15 kDa), cytokines, and an array of toxins bound to proteins and/or other large molecules that exist as multimers. It is difficult to remove these compounds from the patient's circulation using sorption therapy without causing other problems.

At present, there are only a limited number of sorption-based blood purification systems available in the U.S. for treatment of hepatic coma. These include: (1) Adsorba column (Gambro, Hechingen, Germany) that contains activated charcoal, and (2) BioLogic-DT System (HaemoCleanse, West Lafayette, Ind.) utilizing a mixture of charcoal, silica and exchange resins. These systems are rarely used clinically due to their unproven efficacy. In Europe, another system known as MARS, utilizing both activated charcoal and exchange resin is currently in clinical studies (Teraklin, Inc., Germany).

Plasma exchange therapy is achieved by plasmapheresis, i.e., removal of the patient plasma and replacement with normal plasma. In acute liver failure, the rationale for using whole plasma exchange is not only to reduce the level of circulating toxins, but also to provide deficient essential factors (e.g., clotting factors) manufactured by the liver. (Sabin S, Merritt J A. Treatment of hepatic coma in cirrhosis by plasmapheresis and plasma infusion (plasma exchange). Ann Int Med 1968; 68: 1-7; Kondrup J, Almdal T, Vilstrup H, Tygstrup N. High volume plasma exchange in fulminant hepatic failure. Intern J Artif Organs 1992; 15: 669-76).

The results of initial uncontrolled trials of whole plasma exchange therapy for patients with viral hepatitis were not encouraging; only transient biochemical and neurological improvements were achieved, but there was no effect on survival. (Lepore M J, Stutman L J, Bonanno C, et al. Plasmapheresis with plasma exchange in hepatic coma. Arch Int Med 1972; 129: 900-07; Inoue N, Yamazaki Z, Yoshiba M, et al. Membrane plasmapheresis with plasma exchange in the treatment of acute liver failure. Artificial Organs 1981; 5 (suppl): 851-853).

With few exceptions (e.g., Munoz S J, Ballas S K, Moritz M J, et al. Perioperative management of fulminant and subfulminant hepatic failure with therapeutic plasmapheresis. Transplant Proc 1989; 21: 3535-36), the situation has not changed over the years. Therapeutic gains with whole plasma exchange therapy were short-lived and seen predominantly in patients with drug-induced liver failure. (Freeman J G, Matthewsson K. Plasmapheresis in acute liver failure. Intern J Artif Organs 1986; 9: 433-38). The overall survival rate in fulminant hepatic failure (FHF) remained well below 50 percent. (Takahashi T, Malchesky P S, Nose Y. Artificial Liver. State of the Art. Dig Dis Sci 1991; 36: 1327-40). In addition, there was a significant complication rate associated with plasma exchange in these patients (˜40 percent). Although in most cases, they were minor, there were also reports of chemical toxicity, viral infections and deaths from lung and brain complications. (Yoshiba M, Inoue N, Sanjo T, et al. Plasmapheresis in acute liver failure, in Plasmapheresis Therapeutic Applications and New Techniques, eds. Y. Nose, P. S. Malchesky, J. W. Smith and R. S. Krakauer, Raven Press, New York, 1983; pp. 399-406; Brunner G, Losgen H. Benefits and dangers of plasma exchange in patients with fulminant hepatic failure, in Therapeutic Plasinapheresis, VI Therapeutic Plasmapheresis, VI, eds. T. Oda, Y. Shiokawa and N. Inoue, ISAO Press, Cleveland, 1987; pp. 187-191).

Nonetheless, interest in treating FHF with plasma exchange continues. Tygstrup et al. investigated the effect of repeated, high volume plasma exchange in 11 FHF patients. (Tygstrup et al., High volume plasma exchange in fulminant hepatic failure. Intern J Artif Organs 1992; 15: 669-76). On average, 2.6 exchanges were performed on 3 consecutive days, each with a mean volume equal to 16% of the body weight. All 5 patients with acetaminophen-induced FHF survived. Even though the remaining 6 patients died, it is worth noting that they remained stable for a mean of 6.9 days after initiating plasma exchange.

Despite limitations, plasma exchange continues to be the most frequently used method of liver support in patients with FHF. However, it remains impractical because during conventional plasma exchange therapy, up to 20 L (˜40 units) of plasma is removed from the patient and replaced with equal amounts of fresh frozen plasma (FFP) obtained from as many as 100 donors. (Inoue N, et al. Membrane plasmapheresis with plasma exchange in the treatment of acute liver failure. Artificial Organs 1981; 5 (suppl): 851-853). Because of the large amount of FFP needed, complications resulting from massive plasma transfusion, shortage of plasma donors, and high cost, this mode of therapy is rarely used in liver failure patients.

An important need exists in the art to provide an effective blood purification therapy to patients with acute liver failure and other diseases/conditions resulting in accumulation of toxic substances in the circulating blood that is effective and obviates the above-mentioned limitations.

### SUMMARY OF THE INVENTION

The present invention relates to a method of blood purification therapy using selective plasma exchange. In particular, selective plasma exchange therapy (SEPET), in accordance with the present invention, involves replacing a specific plasma fraction of a patient's blood serum with an about equal volume of a plasma substitute suitable for use in a human. Optimally, in any useful blood purification system, plasma exchange therapy included, not all plasma components, should be removed from the patient's blood; many plasma components are beneficial. Consequently, it is a desideratum that those components that are toxic to internal organs, to the central nervous system and to other tissues be removed from the blood, while keeping many beneficial components. During blood purification therapy in accordance with the present invention, this is achieved with efficiency comparable only to high volume total plasma exchange, but with lower costs and health risks to the patient.

In particular, the present invention is directed to a method of removing from a patient's blood a specific plasma fraction containing substances (including toxic substances) within a specific molecular weight range. The method involves attaching to the blood stream of the patient, via catheter means inserted into a blood vessel, a blood perfusion means for extracorporeal blood circulation. Whole blood is removed from the blood stream of the patient and by the blood perfusion means is conveyed to, and circulated through, a selective filtration means, in which filtration of the blood plasma is conducted at a first rate of about 1 to about 20 mL/min for a period of about 1 to about 24 hours. Simultaneously, the patient is infused with a plasma substitute at a second rate about equal to the first rate. The blood plasma, minus the specific plasma portion filtrate, and the blood cells are returned to the patient's blood stream.

An inventive plasma purification apparatus for performing the inventive method is also provided. The apparatus includes a blood perfusion means 200 for extracorporeally circulating a patient's 1 blood. The blood perfusion means includes a first catheter means 210 adapted to attach the blood perfusion means to the patient's blood stream and for providing egress for the patient's blood from the blood stream; and a second catheter means 220 adapted to attach the blood perfusion means to the blood stream and for returning the patient's filtered blood to the blood stream. In one preferred embodiment, the first catheter means and the second catheter means are combined in a double-lumen catheter. The blood perfusion means also includes a first tubing means 230 for conveying the patient's blood flowing from the first catheter means 210; and a second tubing means 240 for conveying the patient' filtered blood to the second catheter means 220.

The blood perfusion means 200 also includes at least one plasma filtration cartridge 300 for filtering the patient's blood; the plasma filtration cartridge is enclosed by a housing 310, and has within the housing, an inner compartment 320 and an outer compartment 330. The inner compartment and the outer compartment are separated by a semipermeable membrane 340 for removing a specific plasma fraction 10 of interest, the semipermeable membrane having a retention coefficient of about 0.50 to about 1.00 for blood plasma constituents with molecular weights greater than a molecular weight of interest, for example, for constituents having molecular weights greater than about 60 kDa to greater than about 200 kDa, which typically, but not necessarily, corresponds to nominal porosities of about 60 kDa to about 200 kDa. The plasma filtration cartridge 300 is adapted for filtering at a rate of about 1 to about 20 mL/min for a period of about 1 to about 24 hours. The plasma filtration cartridge 300 includes an inlet port 350 in the housing for receiving blood flowing from the first tubing means 230 and conveying the blood into the inner compartment 320; a first outlet port 360 in the housing for conveying filtered blood from the inner compartment 320 to the second tubing means 240; and a second outlet port 370 in the housing 310 for conveying a plasma filtrate comprising the specific plasma fraction 10 from the outer compartment 330 for discard, or optionally, for further adsorption 500 of toxic substances in the specific plasma fraction. A reservoir 400 for containing the plasma substitute can optionally be contained within the blood perfusion system of the blood purification apparatus, or alternatively can be separate from it, e.g., an infusion bag completely separate from the apparatus itself.

The blood perfusion means includes a first pump 250 for propelling the patient's blood through the first tubing means 230 from the first catheter means to the inlet port 350 and through the plasma filtration cartridge 300. The first pump 250 is a pump adapted to provide a preselected steady flow rate, e.g., a roller pump. In accordance with the present invention, the first pump 250 can be positioned at any convenient location along the first tubing means 230, between the first catheter means and the inlet port 350 of the plasma filtration cartridge 300. In accordance with the inventive method, the preselected steady flow rate of the first pump 250 is preferably set at a flow rate between about 100 and about 200 mL/min.

The blood perfusion means also includes a second pump 260 for regulating the transmembranous pressure across the semipermeable membrane 340 and determining the rate of plasma exchange. The second pump 260 is a pump adapted to provide a preselected steady flow rate, e.g., a roller pump. In accordance with the present invention, the second pump 260 can be positioned at any convenient location along the third tubing means 380, between the second outlet port 370 and, either a receptacle 600 and/or a plasma sorption means 500. In accordance with the inventive method, the preselected steady flow rate of the second pump 260 is preferably set at a flow rate between about 1 and about 20 mL/min.

It is a benefit of the present inventive method and plasma purification apparatus that a practical blood purification therapy is provided that involves relatively low-volumes of plasma exchange, compared to previously known methods. Thus, the difficulties, expense, and health risks involved in using large quantities of donor plasma as in current methods of blood purification therapy are minimized. The present invention thus provides useful and effective therapy for patients with liver failure, kidney failure, hypercholesterolemia, amyloidosis, sepsis, and inflammatory conditions, such as rheumatoid arthritis.

### BRIEF DESCRIPTION OF THE DRAWING

FIG. 1 depicts a schematic representation of one embodiment of selective plasma exchange therapy in accordance with the present invention. The blood of the patient 1, containing the specific plasma fraction 10, containing all substances with MW from about 1 Dalton up to about 60 kDa to about 200 kDa, depending on the nominal porosity and/or the retention coefficient of the semipermeable membrane 340, is removed and circulated by blood perfusion means 200 through a plasma filtration cartridge 300, and the specific plasma fraction 10 is removed from the second outlet port 370 and replaced with an about equal volume of a plasma substitute 410. FIG. 1 shows an embodiment that includes an optional reservoir 400 for containing the plasma substitute 410, such as, but not limited to, normal whole plasma (e.g., fresh frozen plasma [FFP] previously obtained from human donors). Optionally, a plasma sorption means 500 is included in the system for further adsorption of toxic substances in the specific plasma fraction 10, and the embodiment represented in FIG. 1 also comprises an optional receptacle 600 for collecting the specific plasma fraction 10 for discard.

### DETAILED DESCRIPTION OF THE PREFERRED EMBODIMENTS

The concept of selective plasma exchange therapy (SEPET) is based on knowledge that in many diseases and pathological conditions in human patients, including but not limited to liver failure, toxic substances that accumulate in the blood and cause specific symptoms and/or disease complications are well characterized in terms of their chemical structure and formula or molecular weights. For example, many, if not all, known toxins that accumulate in the blood of a human patient as a result of liver failure, and which can damage brain, liver and other vital organs, are substances smaller than about 100 kDa.

In normal healthy individuals, each plasma component occurs within a range of concentration (e.g., albumin 3.2-4.8 g/dL; bilirubin 0.1-1.0 mg/dL, sodium cation 136-145 mEq/L, etc.), depending on numerous physiological factors (e.g., age, sex, diet, feeding schedule, time of the day or night, presence of stress, etc.). That is why the results of blood tests are typically reported as “above the upper normal level” or “below the lower normal level”. Whether therapeutic intervention is required in response to a particular abnormal value for a given serum component is understood by the skilled practitioner. For example, a patient may have abnormally high levels of blood cholesterol and LDL and, therefore, may be at risk of developing atherosclerosis and suffering from heart attack in the future, but because of chronic liver disease, the patient may have contraindication to certain medications that are available to lower blood lipids. Thus, conventional pharmaceutical treatment may not be prescribed. On the other hand, as an example, very low blood potassium levels may require immediate intravenous administration of K⁺, because of the risk of developing life-threatening cardiac arrhythmia.

Whether treatment using the inventive method and blood purification apparatus is indicated for a patient by the accumulation of one or more toxic serum components outside an acceptable normal range can readily be determined by the skilled practitioner. For example, patients experiencing liver failure, kidney failure, or severe inflammatory responses, such as, but not limited to, rheumatoid arthritis or glomerulonephritis, can be effectively treated by the inventive method and system to remove from their serum dangerous concentrations of toxic substances, generally having molecular weight from about 1 Dalton up to about 200 kDa, and more typically up to about 100 kDa, that can injure the brain, liver, kidneys and other organs. Such toxic substances include, but are not limited to, ammonia, mercaptans, phenols, bilirubin, bile acids, aromatic amino acids, lactic acid, urea, uric acid, proinflammatory cytokines (e.g., tumor necrosis factor [TNF]-α, interleukin [IL]-1, IL-6, IL-8, IL-12, or leukemia inhibitory factor [LIF]) and liver cell growth inhibitors (e.g., transforming growth factor [TGF]-β1).

For purposes of the present invention, the term “molecular weight” (MW) is used to encompass both the molecular weight of a molecular substance and the formula weight of an ionic substance.

To avoid infection to the patient, it will be amply apparent to the skilled practitioner that the steps of the inventive method are preferably executed using known aseptic techniques, and the equipment employed, including the inventive blood purification apparatus, should be sterile. Typically, to keep the blood from clotting, anticoagulant medication, at a dose well known to the skilled practitioner (e.g., as administered in plasmapheresis), is administered to the patient intravenously during execution of the inventive method.

The inventive method involves attaching to a patient's blood stream a blood perfusion 200 means for circulating the patient's blood extracorporeally. Typically, attachment to the patient is transvascular, e.g., by way of a vascular catheter, port, or stent, or other well known first “catheter means” 210 of connecting a patient's blood stream, via a vein or artery, to an extracorporeal tube (i.e., first tubing means 230) removing blood from the patient's blood stream and conveying it into the blood perfusion means 200, thereby allowing blood to flow from the patient into the blood perfusion means 200.

The blood perfusion means 200 can be any known for the purpose of extracorporeal blood circulation. For example, a kidney dialysis machine can be employed. Such machines are commercially available (e.g., Gambro BCT [model PRISMA], B. Braun Medical Inc. (Diapact CRRT; Dialog], Fresenius USA (Fresenius 2008H and 2008K), and Baxter), or can be constructed using known technology. Alternatively, an apparatus other than a kidney dialysis machine can be employed as the blood perfusion means, with or without integrated blood anticoagulation and accessory elements such as pumps, pressure gauges, and the like.

“Tubing means” is a term for any sterilizable flexible hollow tubing, such as but not limited to, silicone or polyvinyl tubing, that can be used for conveying blood, without toxic effect and aseptically. For the purposes of the present invention, a tubing means can be a single tubing segment having a first end and a second opposite end, but, within “tubing means” are also encompassed linked multiples of such tubing segments and any flanges, connectors, adaptors, bubble traps, valves, or the like, that are commonly used to link such tubing segments to each other or to other structures in an apparatus, such as but not limited to, catheters or ports (e.g., inlet or outlet ports).

The skilled artisan can construct the blood perfusion means 200 with one or more modes of operation. Only a single mode of operation facilitating whole blood perfusion and removal of whole plasma and/or plasma fraction is needed, and thus, a simplified set of software controls, safety features, and tubing can be employed

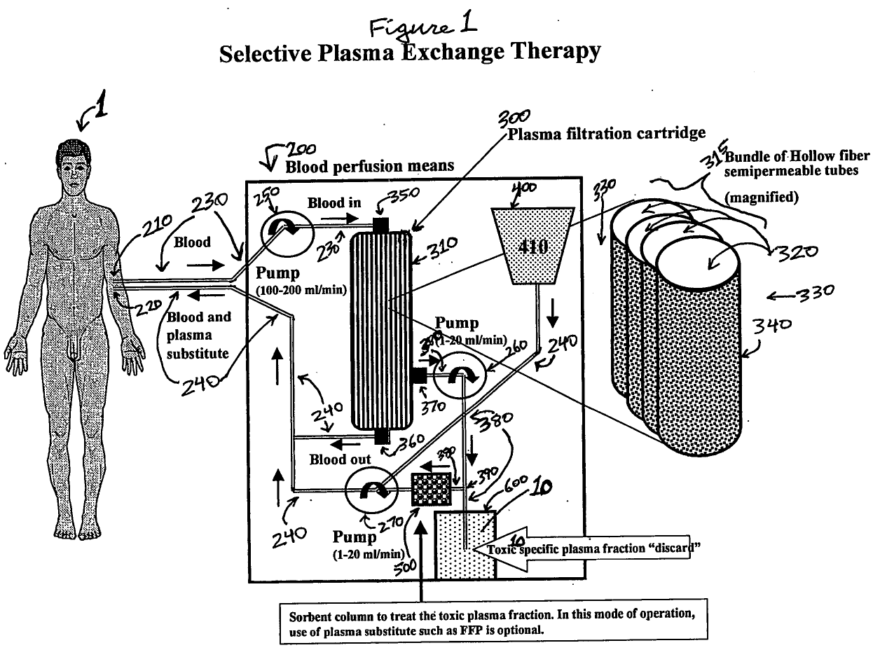

Filtering the blood is accomplished by employing a selective filtration means, for example, but not limited to, a plasma filtration cartridge 300, comprising a semipermeable membrane 340 having a retention coefficient of about 0.50 to about 1.00 for blood plasma constituents greater than a molecular weight of interest, about 60 kDa to about 200 kDa, typically, but not necessarily corresponding to nominal porosities within a range of about 60 kDa to about 200 kDa. Preferably, the semipermeable membrane has a retention coefficient of about 0.50 to about 1.00 for blood plasma constituents with molecular weight greater than about 200 kDa; more preferably, the semipermeable membrane has a retention coefficient of about 0.50 to about 1.00 for blood plasma constituents with molecular weight greater than about 80 kDa to about 150 kDa, typically, but not necessarily, corresponding to nominal porosities within a range of about 80 kDa to about 150 kDa; and most preferably, the semipermeable membrane has a retention coefficient of about 0.50 to about 1.00 for blood plasma constituents with molecular weight greater than about 90 kDa to about 110 kDa, for example, greater than about 100 kDa, typically, but not necessarily, corresponding to a nominal porosity within a range of about 90 kDa to about 110 kDa (e.g., having a nominal porosity about 100 kDa). The semipermeable membrane 340 can be configured in known forms including but not limited to hollow fiber cartridges such as hemofilters, plasma separators, and cell culture devices, for example as shown in FIG. 1, made of any suitable semipermeable membrane material as described above. The semipermeable hollow fiber membrane is manufactured by known techniques (e.g., hot extrusion and use of the spinnerets) and made from known materials, typically comprising a polymeric substance such as, but not limited to, cellulose acetate, polysulfone, modified polysulfone (e.g., polyarylether sulfone, or the like), polyvinylpyrrolidone, polivinylidene difluoride, silicone, polyacrylonitrile, or the like.

The fluid stream that passes through the semipermeable membrane is called “permeate,” and the stream that is retained or rejected by the membrane is termed “retentate.” “Pennselectiyity” is defined as the degree by which the membrane is selectively permeable to the species to be separated. A common measure of the membrane permselectivity in liquid-phase applications is “rejection” or “retention coefficient,” which is equal to the difference between feed and permeate concentrations divided by the feed concentration, expressed as a fraction or percentage.

An example of a useful selective filtration means is a plasma filtration cartridge 300 with desired nominal porosity facilitating removal of the specific plasma fraction within the specific molecular weight range. The “nominal porosity” is the mean pore size of the semipermeable membrane (e.g., as stated by the manufacturer). Generally, the nominal porosity is stated within a standard deviation of about 10%. However, a manufacturer-stated nominal porosity, e.g., 100 kDa, for a semipermeable membrane may not correspond to a retention coefficient of about 0.50 to about 1.00 for blood plasma constituents with molecular weight greater than, e.g., 100 kDa, due to chemical factors, such as, hydration state of the semipermeable membrane, net charge of blood plasma constituents, the presence of multimeric or otherwise complexed plasma constituents, and the like. For purposes of the present invention, the retention coefficient is the property of the semipermeable membrane that is most important, rather than its nominal porosity.

A useful embodiment of a plasma filtration cartridge 300 contains a bundle of hollow fibers 315 (i.e., hollow tubes with wall thickness of about 30 to about 200 microns and an internal diameter of about 100 to about 1000 microns) with walls made of a semipermeable membrane 340. In the bundle, containing about 200 to about 2000 hollow fibers, each typically about 10 cm to about 25 cm in length, the hollow fibers can be unwoven, woven, or in another configuration, such as in a spiraling configuration. The bundle of hollow fibers is enclosed in a rigid housing 310 (e.g., made of a rigid plastic or metallic material), having an inlet port 350, a first outlet port 360 to facilitate blood perfusion through the hollow fibers, and a second outlet port 370, for the recovery of the specific plasma fraction 10 filtered through the semipermable membrane 340. (A typical plasma filtration cartridge 300 is sometimes manufactured with an additional sideport for other applications, but this sideport, if present, is not needed for the present inventive method or apparatus, and it can be kept closed). When the second outlet port 370 is opened, plasma can be collected due to the presence of positive transmembrane pressure generated during whole blood perfusion. In one embodiment of a selective filtration means, one of the widely used hollow fiber plasma separators that is available commercially (e.g., Plasmaflo AP-05H [L], by Asahi Medical Co., Ltd., Japan; distributed in the United States by Apheresis Technologies, Inc.), can be modified, in accordance with the present invention, so that it is manufactured to have hollow fibers comprising semipermeable membranes having the nominal porosity as described hereinabove. The position of the inlet port and first and second outlet ports on the housing is not critical; they may be placed as shown in FIG. 1, or in any other suitable position on the housing 310.

Emerging from the second outlet port 370, the specific plasma fraction 10 is further conveyed by a third tubing means 380 attached to the second outlet port 370. The specific plasma fraction 10 is optionally conveyed by the third tubing means 380 to, and collected in, a receptacle 600, for discard.

Alternatively and optionally, the specific plasma fraction 10 can be conveyed by the third tubing means 380 to an enclosed plasma sorption means 500. The plasma sorption means 500, can be any known, such as cartridge(s) containing activated charcoal, exchange resin and/or polymeric sorbent(s), adapted for receiving the specific plasma fraction 10 conveyed by the third tubing means 380, for adsorbing a toxic substance in the specific plasma fraction 10, and for releasing adsorbed plasma filtrate, purified of toxic substances, to the second tubing means 240 as a plasma substitute 410, for reconstitution with the purified blood (now minus the specific plasma fraction) for return to the patient's 1 blood stream, in accordance with the inventive method., or optionally to a receptacle 510 (not shown in FIG. 1). In this embodiment, it is optional to also use another plasma substitute 410, such as fresh frozen plasma (FFP).

In some embodiments, both the receptacle 600 for receiving the filtered specific plasma fraction 10 for discard, and the plasma sorption means 500 can be present, with a valve 390 placed in the third tubing means 380 for directing, at will, the flow in the third tubing means either to the receptacle 600 or to the plasma sorption means 500.

Some embodiments of the inventive blood purification apparatus have more than one plasma filtration cartridge in series. For example, a plasma filtration cartridge containing semipermeable membranes with a retention coefficient of about 0.50 to about 1.00 for blood plasma constituents with molecular weight greater than about 100 kDa, can further be linked by a fourth tubing means from its first outlet port 360 to the inlet port 350 of a second plasma filtration cartridge of similar structure but containing semipermeable membranes with a retention coefficient of about 0.50 to about 1.00 for blood plasma constituents with molecular weight greater than about 80 kDa. Thus, some embodiments of the inventive blood purification apparatus can have as many as five or more plasma filtration cartridges in series, with descending nominal porosities and/or retention coefficients in succession. In such embodiments, the second tubing means connects the first outlet port 360 of the last plasma filtration cartridge in the series to the second catheter means 220.

In another embodiment of the inventive method, filtering the blood involves pumping the whole blood into a spinning “donut-shaped” loop of a cell separator. In general, a cell separator works either by spinning the blood at high speed to separate the cells from the fluid (e.g., SPECTRA Apheresis System by Gambro BCT), or by passing the blood through a membrane with pores so small that only the fluid part of the blood can pass through. Thus in accordance with the present invention, selective filtration means for filtering the blood can be achieved if the spinning loop of the cell separator is made of a semipermeable membrane having a nominal porosity as described hereinabove. Still another possibility is to separate whole plasma using, for example, Gambro's SPECTRA and then perfuse whole plasma through a hollow-fiber plasma separation cartridge.

In accordance with the inventive method, the selective filtration means are employed for removing a specific plasma fraction 10 from the blood plasma. For purposes of the present invention the “specific plasma fraction” of the patient's blood serum is that fraction of the plasma constituents with molecular weight range from about 1 Dalton (Da) up to about 200 kDa, more preferably from about 1 Dalton up to about 150 kDa, and most preferably from about 1 Dalton up to about 100 kDa. But other useful embodiments of a specific plasma fraction can be selected, including the fraction of the serum containing constituents from about 1 Dalton up to about 80 kDa, or from about 1 Dalton up to about 60 kDa.

The specific plasma fraction 10 includes proteins (e.g., albumin, globulins, complement, blood clotting factors, and the like), other organic molecules such as amino acids, hormones (e.g., insulin, glucagon, parathormone, thyroid hormones, sex hormones, and the like), enzymes (e.g., trypsin, ribonucleases, cytochrome C), cytokines, growth factors, and other groups or classes of organic substances, including but not limited to, sugars (e.g., glucose) and other carbohydrates, salts, bile acids, lipids, vitamins (e.g., Vitamin B₁₂), urea, uric acid, creatinine, ketones, bilirubin, phenols, ethanol, and mercaptans. The specific plasma fraction also can contain a plethora of inorganic chemical substances, including, but not limited to dissolved gases (e.g., oxygen, carbon dioxide, dinitrogen, nitrous oxide, nitric oxide, xenon, neon, hydrogen, helium, ammonia, hydrogen sulfide), and inorganic ions, such as, but not limited to proton, hydronium, hydroxide, chloride, phosphate, bisphosphate, carbonic acid, carbonate, bicarbonate, sulfate, sulfide, selenide, selenate, Na⁺, K⁺, Ca²⁺, Mg²⁺, Fe²⁺, Zn²⁺, Cu²⁺ and the like. The specific plasma fraction can also contain “composite substances”, i.e., complexes of various organic substances, which may also contain inorganic substances.

Simultaneously with the step of filtering the blood, the patient is infused transvascularly (e.g., intravenously) with a plasma substitute 410 at a second rate about equal to the first rate. In accordance with the present invention a “plasma substitute” is a pharmaceutically acceptable aqueous solution (e.g., pH, osmotic strength and electrolyte constituents resembling normal plasma conditions). Preferably, the plasma substitute also contains a normal concentration of albumin, and, most preferably, at least a normal, healthy set of serum peptide components within a molecular weight range from the size of the smallest dipeptide up to about 200 kDa, or up to about 150 kDa, or up to about 100 kDa, or up to about 80 kDa, or up to about 60 kDa. A molecular weight range is preferably chosen that is the same as the specific molecular weight range of the specific plasma fraction that is chosen. The plasma substitute is formulated to be pharmaceutically acceptable for intravascular delivery to the patient. For example, in acccordance with the invention, the plasma substitute can be (1) normal whole plasma from human donors (e.g., fresh or fresh frozen whole plasma [FFP]); (2) a plasma product prepared from normal whole human plasma containing all, or less than all, of the original components of whole plasma, but which preferably, contains a normal concentration of albumin, and, most preferably, at least a normal, healthy set of serum peptide components within a molecular weight range from the size of the smallest dipeptide up to about 200 kDa, or up to about 150 kDa, or up to about 100 kDa, or up to about 80 kDa, or up to about 60 kDa (a molecular weight range is preferably chosen that is within the specific molecular weight range of the specific plasma fraction that is selected); (3) a synthetic product mimicking the serum fraction containing, preferably, a normal concentration of albumin, and, most preferably, at least a normal, healthy set of serum peptide components within a molecular weight range from the size of the smallest dipeptide up to about 200 kDa, or up to about 150 kDa, or up to about 100 kDa, or up to about 80 kDa, or up to about 60 kDa (a molecular weight range is preferably chosen that is within the same range as the specific molecular weight range of the specific plasma fraction that is selected); or (4) a combination of any of (1), (2), or (3). The plasma substitute can also contain additional components, i.e., in addition to the electrolytes, albumin and other peptides described above, for example, glucose and/or non-peptide hormones, to replenish as much as possible all the serum components necessary for physiologic stability of the patient, which are removed from the blood in the specific plasma fraction.

The plasma substitute 410 can be infused into the patient via a valve placed at any point in the second tubing means, or via the second catheter means, or at any other suitable intravenous injection site on the body of the patient via a third catheter means. Optionally, a third pump 270, at the same preselected steady flow rate setting as the second pump 260, can be placed at any convenient location along the second tubing means 240, between a reservoir 400 containing the plasma substitute 410 and the second catheter means.

Alternatively, the plasma substitute 410 can include together with any of the aforementioned plasma substitutes (1)-(4), a plasma fraction of the patient's own serum, which has been purified by adsorption to remove toxic components.

In accordance with the inventive method, for patients, such as liver failure patients, low-volume selective plasma exchange therapy is carried out at a preselected filtration rate of about 1 to about 20 mL/min, and more preferably at a rate of about 1 mL/min to about 10 mL/min, and even more preferably at a rate of about 5 mL/min to about 7 ml/min. The rate is controlled by the setting of the steady flow rate of the second pump 260.

The period of conducting selective plasma exchange therapy in accordance with the invention is for a period sufficient to bring blood levels of toxic plasma constituents that need to be removed to a concentration reduced by at least 50% and/or when desired therapeutic effects are noted (e.g., improvement in coagulopathy, improvement in neurological status, improvement in specific blood parameters such as lowering of bilirubin, ammonia, merkaptans, phenols, bile acids, aromatic amino acids, tumor necrosis factor alpha, transforming growth factor beta, interleukin 6, and the like). Typically, this can be for a period of about 1 hour to about 24 hours, more preferably for a period of about one hour to about 6 hours, and most preferably for a period of about 4 to about 6 hours, using selective filtration means for removing the specific plasma fraction as described herein. Selective plasma exchange therapy, in accordance with the invention, can be conducted continuously and/or repeatedly, i.e., during sequential sessions of therapy, as needed.

The removed plasma fraction is replaced with an equal amount of the plasma substitute. FIG. 1 illustrates schematically the inventive method applied to a patient for the purpose of selective plasma exchange therapy.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are intended to be embraced therein.

1. A method for removing from a patient's blood a specific plasma fraction containing substances within a specific molecular weight range, the method comprising the steps of:
(a) attaching to the blood stream of the patient a blood perfusion means for extracorporeal blood circulation, the blood perfusion means comprising a selective filtration means;
(b) removing blood from the blood stream of the patient, and conveying the blood extracorporeally to the selective filtration means;
(c) filtering the blood with the selective filtration means, the selective filtration means being adapted for removing the specific plasma fraction from the blood, at a first rate of about 1 to about 20 ml/min for a period of about 1 to about 24 hours;
(d) returning the filtered blood to the patient, minus the specific plasma fraction; and
(e) simultaneously infusing the patient with a plasma substitute at a second rate about equal to the first rate.

2. The method of claim 1, wherein the specific molecular weight range is about 1 Da to about 200 kDa.

3. The method of claim 2, wherein the specific molecular weight range is about 1 Da to about 150 kDa.

4. The method of claim 3, wherein the specific molecular weight range is about 1 Da to about 100 kDa.

5. The method of claim 4, wherein the specific molecular weight range is about 1 Da to about 80 kDa.

6. The method of claim 5, wherein the specific molecular weight range is about 1 Da to about 60 kDa.

7. The method of claim 1, wherein the first rate is about 1 to about 10 mL/min.

8. The method of claim 1, wherein the period is about 1 to about 6 hours.

9. The method of claim 1, wherein the plasma substitute is selected from the group consisting of
(a) normal whole plasma from human donors;
(b) a plasma product prepared from normal whole human plasma;
(c) a synthetic product mimicking the serum fraction; and
(d) a combination of any of (a), (b), or (c).

10. A plasma purification apparatus, comprising:
a blood perfusion means for extracorporeally circulating a patient's blood, said blood perfusion means further comprising
(i) a first catheter means adapted to attach the blood perfusion means to the patient's blood stream and for providing egress for the patient's blood from the blood stream; and
(ii) a second catheter means adapted to attach the blood perfusion means to the blood stream and for returning the patient's filtered blood to the blood stream;
(iii) a first tubing means for conveying the patient's blood flowing from the first catheter means;
(iv) a first pump for propelling the patient's blood through the first tubing means at a first preselected steady flow rate, the first pump being positioned at a location on the first tubing means;
(v) a second tubing means for conveying the patient' filtered blood to the second catheter means;
(vi) at least one plasma filtration cartridge for filtering the patient's blood, the plasma filtration cartridge enclosed by a housing, and having within the housing, an inner compartment and an outer compartment; the inner compartment and the outer compartment being separated by a semipermeable membrane having a retention coefficient of about 0.50 to about 1.00 for blood plasma constituents with molecular weights greater than a molecular weight of interest, for removing a specific plasma fraction, said plasma filtration cartridge being adapted for filtering at a rate of about 1 to about 20 mL/min for a period of about 1 to about 24 hours, said plasma filtration cartridge comprising:
(a) an inlet port in the housing for receiving blood flowing from the first tubing means and conveying the blood into the inner compartment;
(b) a first outlet port in the housing for conveying filtered blood from the inner compartment to the second tubing means; and
(c) a second outlet port in the housing for conveying a plasma filtrate from the outer compartment
(vii) a third tubing means; and
(viii) a second pump for regulating the transmembranous pressure across the semipermeable membrane, the second pump being adapted for pumping at a second preselected steady flow rate and being positioned at a location along the third tubing means.

11. The apparatus of claim 10, wherein the first catheter means and the second catheter means are combined in a double-lumen catheter.

12. The apparatus of claim 10, further comprising an enclosed plasma sorption means joined to the second outlet port by the third tubing means, the plasma sorption means adapted for receiving the plasma filtrate conveyed by the third tubing means, for adsorbing a toxic substance in the plasma filtrate, and for releasing adsorbed plasma filtrate to a receptacle.

13. The apparatus of claim 10, wherein the molecular weight of interest is about 200 kDa, or less.

14. The apparatus of claim 13, wherein the molecular weight of interest is about 150 kDa, or less.

15. The apparatus of claim 14, wherein the molecular weight of interest is about 100 kDa, or less.

16. The apparatus of claim 15, wherein the molecular weight of interest is about 80 kDa, or less.

17. The apparatus of claim 16, wherein the molecular weight of interest is about 60 kDa, or less.
